(19) United States
(12) Patent Application Publication
(10) Pub. No.: US 2006/0141044 A1
(43) Pub. Date: Jun. 29, 2006

(54) Pharmaceutical compositions based on diclofenac derivate

(76) Inventors: Christina Gustafsson, Sodertalje (SE); Sven Morein, Sodertalje (SE)

Correspondence Address: ARENT FOX PLLC, 1050 CONNECTICUT AVENUE, N.W., SUITE 400, WASHINGTON, DC, 20036, US

(21) Appl. No.: 10/560,824

(22) PCT Filed: Jun. 23, 2004
(86) PCT No.: PCT/SE04/01017

§ 371 Date: Dec. 15, 2005

(30) Foreign Application Priority Data
Jun. 25, 2003 (SE) 0301880-1

(51) Int. Cl. A61K 9/14; A61K 31/24

(52) U.S. Cl. 424/489; 514/534; 977/906

(57) ABSTRACT

The present invention relates to particles comprising the drug 2-[2-(nitrooxy)ethoxy]ethyl{2-[(2,6-dichlorophenyl)amino]phenyl}acetate, optionally mixed with one or more surfactant(s) and to a new drug delivery composition comprising said particles optionally in combination with a second drug. Furthermore, the invention relates to processes for preparing said particles and drug delivery composition as well as the use of said composition in the manufacturing of a medicament.

### FIELD OF INVENTION

The present invention relates to particles comprising the drug 2-[2-(nitrooxy)ethoxy]ethyl {2-[(2,6-dichlorophenyl)amino]phenyl} acetate, optionally mixed with one or more surfactant(s) and to a new drug delivery composition comprising said particles optionally in combination with a second drug.

Furthermore, the invention relates to processes for preparing said particles and drug delivery composition as well as the use of said composition in the manufacturing of a medicament.

### BACKGROUND OF THE INVENTION

Non-steroidal anti-inflammatory drugs, commonly and hereafter abbreviated as NSAIDs, are well-known drugs for the treatment of pain and inflammation. One of the major drawback of NSAIDs is that they have severe gastro-intestinal side-effects. Patients undergoing treatment with NSAIDs for a longer period of time, such as naproxen, often experience problems with stomach gastrointestinal side-effects.

Nitrogen oxide donating NSAID drugs (in the following NO-donating NSAIDs), have been found to have an improved side-effect profile, see e.g. WO 94/04484, WO 94/12463, WO 95/09831 and WO 95/30641.

NO-donating NSAIDs are lipophilic drugs with poor aqueous solubility. A biopharmaceutical problem with these drugs is that their absorption from the gastro-intestinal tract (GIT) may be dissolution rate limited, resulting in poor bioavailibility upon oral administration.

An advantageous solution to the problem in handling drugs having a low melting point is forming a Self Emulsifying Drug Delivery System, commonly known as SEDDS, e.g. as described in WO 01/66087. More particularly, the SEDDS is a pharmaceutical composition suitable for oral administration, in the form of an emulsion pre-concentrate, comprising one or more NO-donating NSAID(s); one or more surfactant(s); and optionally an oil or semi-solid fat. The composition forms in-situ an oil-in-water emulsion upon contact with aqueous media such as gastrointestinal fluids. The pre-concentrate emulsion is usually filled into conventional capsules.

Emulsion or preconcentrates are not the preferred compositions in pharmaceutical industry. One drawback may for example be the stability of such formulations. Tablets and capsules are often preferred in view of large scale manufacturing of drug delivery compositions.

Tabletted compositions comprising an oily, sticky component and a method for preparing such compositions are described in WO 99/27912 and WO 99/27913. These documents describe absorption of the oily sticky component into/onto a unsoluble inorganic carrier.

The NO-donating NSAID, or NO-donating diclofenac, 2-[2-(nitrooxy)ethoxy]ethyl{2-[(2,6-dichlorophenyl)amino]phenyl}acetate, hereinafter referred to as the “drug”, has a low solubility in water and a low melting point. Because of the low solubility in water the absorption of the drug from the gastro-intestinal tract (GIT) may be dissolution rate limited.

The low melting point may cause problems during the preparation of conventional pharmaceutical compositions such as granulation of a mixture of the drug with excipients and tablet compression. This is especially true for large-scale production. The high temperature in the tablet press may melt the drug, which then will stick to the tablet punches. One way to avoid this is to “dilute” the active drug with suitable excipients. Large amounts of excipients may in turn result in an unacceptable size of a drug delivery composition such as a tablet or capsule.

The bioavailavility of a drug will improve if the drug is easily released from the composition. The release of the drug is among others, dependent on the size of the drug particles in the composition. The smaller the drug particles the better the drug will be released from the composition. The procedure, where the drug is melted, absorbed/adsorbed into/onto a carrier particle, and then recrystallized, leads to a large decrease in drug particle size. This will in turn increase the release rate of the drug from the composition.

It has surprisingly been found that the problems regarding the low melting point of the drug and low bioavailability can be overcome by the drug delivery composition of the present invention.

### DETAILED DESCRIPTION OF THE INVENTION

2-[2-(nitrooxy)ethoxy]ethyl {2-[(2,6-dichlorophenyl)amino]phenyl}acetate is a lipophilic drug with poor aqueous solubility and a low melting point. Said drug can be classified into class 2 according to the Biopharmaceutical Classification System proposed by Amidon et al. (Pharm. Res. 12 (1995) pp. 413420). Compounds of this class are characterised by their low aqueous solubility but reasonably well permeability. A biopharmaceutical problem with these drugs is that their absorption from the gastro-intestinal tract (GIT) may be dissolution rate limited, resulting in poor bioavailibility upon oral administration. One object of the invention is to provide an oral drug delivery composition with satisfactory bioavailability.

By first melting and subsequently adsorb/absorb the drug onto and/or into water soluble carrier particles, the dissolution rate of the drug is enhanced.

Absorption of the drug into carrier particles may reduce the amount of excipients needed to avoid stickiness.

Further, the water soluble particles will solubilize in the gastro-intestinal tract and thereby enhance the release of the drug form the composition.

Active Drug

The NO-donating NSAID 2-[2-(nitrooxy)ethoxy]ethyl {2-[(2,6-dichlorophenyl)amino]phenyl}acetate as well as processes for preparation thereof is disclosed in WO 95/30641.

The Drug Delivery Composition

It has been found that the lipofilic drug 2-[2-(nitrooxy)ethoxy]ethyl (2-[(2,6-dichlorophenyl)amino]phenyl)acetate can be formulated by melting the drug and adsorb/absorb it onto/into water soluble carriers. Useful carriers for the drug are carrier particles having properties such as large total surface area where it is possible for the lipofilic drug to adsorb and/or absorb onto/into the particles. Also, the particles should be easily soluble in water.

Advantages of this melting procedure is that the adsorbed and/or absorbed and recrystallised drug has a particle size that is much smaller compared to the pure drug. This in turn leads to a considerable increase in dissolution rate. The use of water soluble particles will further increase the dissolution rate, compared to the case when the drug is melted and adsorbed and/or absorbed to particles having a poor solubility in water.

The particles used for the drug delivery composition of the present invention may be porous or non-porous. When non-porous particles are used the drug will only be adsorbed onto the particles. When porous particles are used the drug will be absorber into the particles as well as adsorbed onto the surface of the particles.

Throughout this application the term “particle” includes both non-porous and porous particles, as well as mixtures thereof.

One embodiment of the invention relates to a drug delivery composition comprising 2-[2-(nitrooxy)ethoxy]ethyl{2-[(2,6-dichlorophenyl)amino]phenyl}acetate adsorbed onto non-porous particles.

Another embodiment of the invention relates to a drug delivery composition comprising 2-[2-(nitrooxy)ethoxy]ethyl{2-[(2,6-dichlorophenyl)amino]phenyl}acetate absorbed and adsorbed into and onto porous particles.

A further embodiment of the invention relates to a drug delivery composition comprising 2-[2-(nitrooxy)ethoxy]ethyl {2-[(2,6-dichlorophenyl)amino]phenyl}acetate in melted form absorbed/adsorbed onto/into particles.

The material of the particles used for adsorbing/absorbing the drug may be selected from materials such as mannitol or lactose or mixtures of lactose or mannitol with microcrystalline cellulose, cellulose or starch.

One embodiment of the invention relates to a drug delivery composition according to the present invention whereby the material of the particles is selected from the group consisting of mannitol and lactose, optionally in admixture with one or more substances selected form the group consisting of microcrystalline cellulose, cellulose and starch. Another embodiment of the invention relates to a drug delivery composition according to the present invention whereby the material of the particles is mannitol, particularly granulated pure mannitol such as Pearlitol®.

The particle material used as carrier shall have a particle size between 20 and 500 μm, particularly a size between 50 and 150 μm.

Thus, 95% of the particles used in the composition of the present invention shall have a size in the ranges mentioned above.

One embodiment of the invention relates to the drug delivery composition of the invention wherein the particles have a size between 50 and 500 μm.

Another embodiment of the invention relates to the drug delivery composition of the invention wherein the particles have a size between 100 and 150 μm.

The pore size of the porous particles should be between 10 and 1000 Å, particularly between 20 and 750 Å, and most suitably between 50 and 500 Å.

Thus, 95% of the particles used in the composition of the present invention shall have a pore size in the ranges mentioned above.

One embodiment of the invention relates to the drug delivery composition of the invention wherein the particles have a pore size between 10 and 1000 Å.

Another embodiment of the invention relates to the drug delivery composition of the invention wherein the particles have a pore size between 20 and 750 Å.

The drug may be melted and adsorbed/absorbed onto/into the porous particles either as the sole drug; as a SEDDs formulation; or as a finely dispersed or dissolved drug.

Surfactants

The release rate of the drug from the composition may be influenced by the presence or absence of one or more surfactant(s). It has been shown that the release characteristics can be changed by adding one or more surfactant(s). The rate of release may be increased if a suitable surfactant is present in the drug delivery composition together with the drug.

One embodiment of the invention relates to a drug delivery composition comprising particles comprising 2-[2-(nitrooxy)ethoxy]ethyl {2-[(2,6-dichlorophenyl)amino]phenyl}acetate in admixture with one or more surfactant(s).

Another embodiment of the invention relates to a drug delivery composition comprising a combination of

a) particles comprising 2-[2-(nitrooxy)ethoxy]ethyl{2-[(2,6-dichlorophenyl)amino]phenyl}acetate and one or more surfactant(s), and

b) particles comprising 2-[2-(nitrooxy)ethoxy]ethyl {2-[(2,6-dichlorophenyl)amino]phenyl}acetate without surfactant.

Such a drug delivery composition will give a more advanced release profile of the drug, for example a first rapid onset by release from the particles comprising the drug with one or more surfactant(s) followed by a delayed release from the particles comprising the drug alone.

The wording “surfactant” is defined as surface-active amphiphilic drugs. Suitable surfactants are non-ionic surfactants, for example those containing polyethylene glycol (PEG) chains, particularly block co-polymers such as poloxamers.

One embodiment of the invention relates to a drug delivery composition comprising the drug and one or more surfactant(s) whereby the surfactant(s) is a non-ionic surfactant. Another embodiment of the invention relates to a drug delivery composition comprising the drug and one or more surfactant(s) whereby the surfactant(s) is a poloxamers.

A further embodiment of the invention relates to a drug delivery composition comprising the drug and one or more surfactant(s) whereby one of the surfactants is polyoxyethylene polyoxybutylene block copolymer.

The poloxamers that may be used in the drug delivery composition of the present invention may be selected form the group comprising of Poloxamer 407 (Pluronic F127®), Poloxamer 401 (Pluronic L121®), Poloxamer 237 (Pluronic F87®), Poloxamer 338 (Pluronic F108®), Poloxamer 331 (Pluronic L101®), Poloxamer 231 (Pluronic L81®), tetrafunctional polyoxyethylene polyoxypropylene block copolymer of ethylene diamine, for example Poloxamine 908 (Tetronic 908®), Poloxamine 1307 (Tetronic 1307®), Poloxamine 1107, polyoxyethylene polyoxybutylene block copolymer, for example Polyglycol BM45®. This list should not in any way be considered as exhaustive or limiting the invention.

One embodiment of the invention relates to a drug delivery composition comprising the drug and one or more surfactant(s) whereby the surfactant(s) is selected from the group consisting of Poloxamer 407, Poloxamer 401, Poloxamer 237, Poloxamer 338, Poloxamer 331, Poloxamer 231, Poloxamine 908, Poloxamine 1307, Poloxamine 1107 and polyoxyethylene polyoxybutylene block copolymer, or mixtures thereof.

All surfactants described above are commercially available from for example BASF, Dow Chemicals or Gattefossé. The total amount of surfactant(s) used in the drug delivery composition of the invention may be within a range from 2 mg to 10 g, particularly from 20 to 1000 mg.

The ratio drug:surfactant(s) may vary from 1:0.1 to 1:10 (w/w), particularly from 1:0.3 to 1:3 (w/w).

One embodiment of the invention relates to a drug delivery composition wherein the ratio drug:surfactant(s) is from 1:0.1 to 1:10 (w/w).

Another embodiment of the invention relates to a drug delivery composition wherein the ratio drug:surfactant(s) is from 1:0.3 to 1:3 (w/w).

Further, the drug delivery composition of the present invention may comprise a combination of 2-[2-(nitrooxy)ethoxy]ethyl {2-[(2,6-dichlorophenyl)amino]phenyl}acetate and one or more other active drugs, optionally with one or more surfactant(s).

Preparation of the Particles

The incorporation of the drug onto/into the particles may be accomplished by conventional known methods.

Without Surfactant

The particles comprising the drug may be prepared in different ways, for example by mixing the drug with the particles directly, e.g. in a mortar, and subsequently melting the is drug.

Alternatively, the drug may be melted before mixing with the particles.

Also, the drug may be dissolved in a suitable solvent. The particles may than be added to the particles after which the drug will be absorbed/absorbed. The solvent(s) is then evaporated and the particles are collected.

One embodiment of the invention relates to a process for preparing the particles comprising the drug comprising mixing 2-[2-(nitrooxy)ethoxy]ethyl {2-[(2,6-dichlorophenyl)amino]phenyl}acetate in melted form with particles.

Another embodiment of the invention relates to a process for preparing the particles comprising 2-[2-(nitrooxy)ethoxy]ethyl {2-[(2,6-dichlorophenyl)amino]phenyl}acetate comprising:

a) melting the drug,

b) adding the particles,

c) stirring the obtained mixture,

d) recovering the porous particles comprising the drug.

A further embodiment of the invention relates to a process for preparing the particles comprising 2-[2-(nitrooxy)ethoxy]ethyl {2-[(2,6-dichlorophenyl)amino]phenyl}acetate comprising:

a) mixing the drug with the particle,

b) melting the obtained mixture,

c) stirring the obtained mixture,

d) recovering the particles comprising the drug.

With Sufactant

The drug may be mixed with one or more liquid surfactant(s), and then adsorbed/absorbed onto/into the particles. The surfactant(s) may be in solid or liquid form. If needed the components may be melted before mixing to get a homogeneous mixture of the drug and the surfactant(s) before adding the particles.

One embodiment of the present invention relates to a process for preparing the particles comprising 2-[2-(nitrooxy)ethoxy]ethyl {2-[(2,6-dichlorophenyl)amino]phenyl}acetate and one or more surfactant(s) comprising:

a) melting the drug and the surfactant(s),

b) adding the particles,

c) stirring the obtained mixture,

d) recovering the particles comprising the drug and the surfactant(s),

with a) and b) in optional order.

The drug may optionally be pre-heated before starting the proces.

One embodiment of the invention relates to any of the processes described above wherein the drug in step a) is pre-heated.

Preparation of the Drug Delivery Composition

The particles comprising the drug with or without surfactant(s) may be formulated by mixing the particles comprising the drug, with or without surfactant(s), with pharmaceutically acceptable diluent, excipients and/or inert carriers such as fillers, binders, disintegrants followed by formulation of the obtained mixture into a suitable drug delivery composition.

Examples of suitable drug delivery composition are capsules and tablets. Tablets may be obtained by direct compression or after granulation.

The particles comprising the drug, with or without surfactant(s), may also be used as such, for example in a sachet.

The particles comprising the drug, optionally in admixture with pharmaceutically acceptable diluent, excipients and/or inert carriers may also be suspended in a water solution to form a suspension.

Optionally, the particles comprising the drug, with or without surfactant(s), may be mixed with a second active drug, before formulated into a suitable drug delivery composition.

Example of suitable diluent, excipients and/or inert carriers, but not limited thereto, are collodial silica, sodium stearyl fumarate, magnesium stearate, polyvinyl pyrrolidone such as polyvidon XL, polyvidon K-30, cellulose derivatives such as hydroxypropyl methylcellulose (HPMC), sodium carboxymethylcelulolose (NaCMC), microcrystalline cellulose, mannitol, and lactose.

One embodiment of the present invention relates to a drug delivery composition wherein the particles comprising the drug, optionally in admixture with one or more surfactant(s), are mixed with pharmaceutically acceptable diluent, excipients and/or inert carrier.

Another embodiment of the present invention relates to a drug delivery composition wherein the particles comprising the drug, optionally in admixture with one or more surfactant(s), are formulated into a tablet.

A further embodiment of the present invention relates to a drug delivery composition wherein the particles comprising the drug, optionally in admixture with one or more surfactant(s), are filled into a capsule.

Yet another embodiment of the present invention relates to a drug delivery composition wherein the particles comprising the drug, optionally in admixture with one or more surfactant(s), are suspended in a water solution.

Yet a further embodiment of the invention relates to a process for the preparation of the drug delivery composition comprising;

a) mixing the particles, obtained according to any one of the processes described above, with pharmaceutically acceptable diluent, excipients and/or inert carrier,

b) granulating the obtained mixture with water,

c) drying the granulate,

d) optionally mixing the granulate with further diluent, excipients and/or inert carrier, and

e1) filling the granulate into capsules, or

e2) compressing the granulate into tablets.

The particles, capsules and tablets may be coated by ways well known in the art. The filling into capsules, compressing to tablets and coating should preferably be performed in such a manner that does not substantially influence the release characteristics of the drug delivery composition after administration.

The prepared particles, capsules and tablets may be coated by a conventional film coat or a sugar coat, to obtain an improved appeareance. Suitable layering material for the film coat, but not limited thereto, are derivatives of cellulose such as hydroxypropylmethylcellulose, methylcellulose or ethylcellulose and acrylate-based polymers.

Sugar coating involves successive application of sucrose based solutions to the particles, capsules or tablets.

One embodiment of the invention relates to a drug delivery composition in the form of a capsule and tablet, which is coated.

If the release of the drug is desired in the small intestine, the particles comprising the drug, optionally in admixture with one or more surfactant(s), may be enteric coated.

Another embodiment of the invention relates to a divisable drug delivery composition, for example, a divisable tablet.

The total amount of drug used in the drug delivery composition of the invention may be between 20 mg and 1 g per unit dose, particlularly between 25 and 600 mg, more particularly between 50 and 200 mg.

Use

One embodiment of the invention relates to the drug delivery composition of the present invention, for use in the treatment of pain and/or inflammation.

Another embodiment of the invention relates to the use of the drug delivery composition according to the present invention for the manufacture of a medicament for the treatment of pain and/or inflammation.

A further embodiment of the invention relates to a method of treatment of pain and/or inflammation, comprising administration to a patient in need of such treatment, the drug delivery composition according to the present invention.

In the context of the present specification, the term “therapeutically” and “treatment” includes prevention and prophylaxis, unless there are specific indications to the contrary.

### EXAMPLES

Powders comprising soluble particles comprising the drug, 2-[2-(nitrooxy)ethoxy]ethyl {2-[(2,6-dichlorophenyl)amino]phenyl}acetate, were made by mixing the drug with the particles as described below.

A) The Drug/Pearlitol® 100 SD 1/3

1-g Drug

3 g Pearlitol®

The drug was melted at 75° C. and mixed with Pearlitol® until a homogenous powder was obtained. The powder mixture was allowed to cool while stirring. Subsequently, the powder was filled into hard gelatine capsules.

B) The Drug/Pearlitol® 100 SD 1/2.8

10.5 g Drug

29.5 g Pearlitol®

0.62 g Microcrystalline cellulose

0.63 g Polyvidon XL

0.41 g Polyvidon K-30

0.38 g Collodial Silica

0.20 g Sodium Stearyl Fumarate

11.2 g Water Purified

The drug and Pearlitol® were mixed in an intensive mixer. The mixture was heated to 75° C. under continous mixing until the drug was fully melted. The mixture was cooled to room temperature and the obtained powder was sieved through a 0.355 mm sieve. 37.8 g of the sieved powder was mixed with microcrystalline cellulose, polyvidon XL, and polyvidon K-30 in an intensive mixer. The powder was wet-granulated with a small amount of water. The granulate was dried overnight at 45° C. Collodial silica was added to the dried granulate and the powder was mixed for 5 min. Sodium stearyl fumarate was added to the mixture followed by 1 min of mixing. The granulate was filled into hard gelatin capsules.

Results

The dissolution rate was monitored accordording to USP (paddle method). The dissolution medium had a temperature of 37° C. The media used was 0.01 M HCl(aq), containing 8.8 mg/litre of cetyltrimethylammonium bromide. The increase in absorbance corresponded to the release of the drug from the drug delivery composition.

A) The Drug/Pearlitol™ 1/3. Capsule, 100 mg of Drug

| Time | % Released |
|---|---|
| 5 min | 11.2 |
| 10 min | 37.2 |
| 15 min | 54.2 |
| 30 min | 78.9 |
| 60 min | 94.7 |
| 90 min | 100.2 |

B) The Drug/Pearlitol™ 1/2.8. Capsule, 100 mg of Drug

| Time | % Released |
|---|---|
| 5 min | 12.1 |
| 10 min | 33.4 |
| 20 min | 60.7 |
| 45 min | 79.6 |
| 90 min | 88.14 |

1. A drug delivery composition comprising 2-[2-(nitrooxy)ethoxy]ethyl {2-[(2,6-dichlorophenyl)amino]phenyl}acetate in melted form absorbed/adsorbed onto/into particles.

2. The drug delivery composition according to claim 1 whereby the materials of the particles selected from the group consisting of mannitol and lactose, optionally in admixture with one or more substances selected form the group consisting of microcrystalline cellulose, cellulose and starch.

3. The drug delivery composition according to any one of claims 1 to 2 wherein the particles have a size between 50 and 500 μm.

4. The drug delivery composition according to claim 3 wherein the particles have a size between 100 and 150 μm.

5. The drug delivery composition according to any one of claims 1 to 4 wherein the particles have a pore size between 10 and 1000 Å.

6. The drug delivery composition according to claim 5 wherein the particles have a pore size between 20 and 750 Å.

7. The drug delivery composition according to any one of claims 1 to 6 comprising particles comprising 2-[2-(nitrooxy)ethoxy]ethyl{2-[(2,6-dichlorophenyl)amino]phenyl}acetate in admixture with one or more surfactant(s).

8. The drug delivery composition according to any one of claims 1 to 6 comprising a combination of
a) particles comprising 2-[2-(nitrooxy)ethoxy]ethyl {2-[(2,6-dichlorophenyl)amino]phenyl}acetate and one or more surfactant(s), and
b) particles comprising 2-[2-(nitrooxy)ethoxy]ethyl {2-[(2,6-dichlorophenyl)amino]phenyl}acetate without surfactant.

9. The drug delivery composition according to claims 7 and 8 wherein the surfactant(s) is a non-ionic surfactant.

10. The drug delivery composition according to claim 9 wherein the surfactant(s) is a block co-polymer.

11. The drug delivery composition according to claim 9 wherein the surfactant(s) is a poloxamer.

12. The drug delivery composition according to claims 7 and 8 wherein the ratio drug:surfactant(s) is from 1:0.1 to 1:10 (w/w).

13. The drug delivery composition according to any one of claims 1 to 12 wherein the particles comprising the drug, optionally in admixture with one or more surfactant(s), are mixed with pharmaceutically acceptable diluent, excipients and/or inert carrier.

14. The drug delivery composition according to any one of claims 1 to 13 wherein the particles comprising the drug are formulated into a tablet.

15. The drug delivery composition according to any one of claims 1 to 13 wherein the particles comprising the drug are filled into a capsule.

16. The drug delivery composition according to any one of claims 1 to 13 wherein the particles comprising the drug are suspended in a water solution.

17. The drug delivery composition according to claims 14 and 15, which is coated.

18. The drug delivery composition according to any one of the claims 1 to 17, for use in the treatment of pain and/or inflammation.

19. Use of the drug delivery composition according to any one of the claims 1 to 17 for the manufacture of a medicament for the treatment of pain and/or inflammation.

20. A method of treatment of pain and/or inflammation, comprising administration to a patient in need of such treatment, the drug delivery composition according to any one of the claims 1 to 17.

21. A process for preparing particles comprising 2-[2-(nitrooxy)ethoxy]ethyl{2-[(2,6-dichlorophenyl)amino]phenyl}acetate comprising mixing the drug in melted form with particles.

22. A process for preparing particles comprising 2-[2-(nitrooxy)ethoxy]ethyl {2-[(2,6-dichlorophenyl)amino]phenyl}acetate comprising:
a) melting the drug,
b) adding the particles,
c) stirring the obtained mixture,
d) recovering the porous particles comprising the drug.

23. A process for preparing particles comprising 2-[2-(nitrooxy)ethoxy]ethyl{2-[(2,6-dichlorophenyl)amino]phenyl}acetate comprising:
a) mixing the drug with the particle,
b) melting the obtained mixture,
c) stirring the obtained mixture,
d) recovering the particles comprising the drug.

24. A process for preparing particles comprising 2-[2-(nitrooxy)ethoxy]ethyl{2-[(2,6-dichlorophenyl)amino]phenyl}acetate and one or more surfactant(s) comprising:
a) melting the drug and the surfactant(s),
b) adding the particles,
c) stirring the obtained mixture,
d) recovering the particles comprising the drug and the surfactant(s),
with a) and b) in optional order.

25. The processes according to any one of claims 22 to 24 whereby the drug in step a) is pre-heated.

26. A process for the preparation of the drug delivery composition according to claims 14 or 15 comprising;
a) mixing the particles, obtained according to any one of the processes of claims 21 to 25, with pharmaceutically acceptable diluent, excipients and/or inert carrier,
b) granulating the obtained mixture with water,
c) drying the granulate,
d) optionally mixing the granulate with further diluent, excipients and/or inert carrier, and
e1) filling the granulate into capsules, or
e2) compressing the granulate into tablets.
